(19) United States
(12) Patent Application Publication
(10) Pub. No.: US 2011/0236363 A1
(43) Pub. Date: Sep. 29, 2011

(54) SYSTEM AND METHOD FOR PRODUCING T CELLS

(76) Inventors: Lung-Ji Chang, Gainesville, FL (US); Ekta Samir Patel, Gainesville, FL (US)

(21) Appl. No.: 13/062,570

(22) PCT Filed: Sep. 11, 2009
(86) PCT No.: PCT/US09/56739

§ 371 Date: May 26, 2011

Related U.S. Application Data
(60) Provisional application No. 61/096,240, filed on Sep. 11, 2008.

(51) Int. Cl. A61K 35/28; C12N 5/0783; C12N 5/073; A61P 35/00; A61P 35/02

(52) U.S. Cl. 424/93.71; 435/377; 435/373; 435/354; 435/353; 435/352; 435/325

(57) ABSTRACT

Disclosed herein is a system and method for producing T cells from stem cell populations. Specifically exemplified herein is a culture system and method that produces CD4 cells and/or T cell subtypes from a CD4 lineage using a sample of hematopoietic stem cells. Adult hematopoietic precursor/stem cells (HPC) are progenitors to all lineages of immune cells. There has been limited success in generating functional CD4 T cells with this convenient culture system. Also disclosed herein is a novel stromal cell line expressing DL1, interleukin-7 (IL-7), and FMS-like tyrosine kinase 3 ligand (Flt3-L). This improved culture system can greatly facilitate the study of late T cell development and enables immunotherapeutic applications.

### CROSS-REFERENCE TO RELATED APPLICATIONS

This application is related to U.S. Provisional Application 61/096,240 filed Sep. 11, 2008 to which priority is claimed under 35 USC 119.

### STATEMENT OF GOVERNMENT SUPPORT

This invention was made with Government support under Agreement NIH grant HL59412. The Government has certain rights in the invention.

### INTRODUCTION

T cells play an important role in the establishment of the mammalian immune system. The immune system often fails to function properly in patients suffering from chronic infections or cancer (1). Large-scale production of T cells with the aim for the treatment of infections and cancer has been of continuous interest. Autologous transfer of in vitro expanded antigen-specific lymphocytes is challenged by limited sources of healthy and functional T cells (2). Adoptive transfer of allogenic antigen specific effector T cells is limited by availability of such reactive T cells and faces the problem of graft-versus-host disease (GVHD) (3). Hence, producing large number of antigen specific T cells from adult human bone marrow (BM) derived CD34 hematopoietic precursor/stem cells (HPC) in vitro could help overcome some of the limitations described above.

Previously established in vitro culture systems for producing human T lymphocytes such as thymus organ cultures and three-dimensional matrices of epithelial cells are labor intensive and difficult to manipulate (4-6). These in vitro culture systems have demonstrated early T cell differentiation from embryonic stem cells of mouse and human origins (7, 8). Recently, a simpler T cell development culture system has been reported that employs mouse fetal stromal cells engineered to express the Notch ligand Delta-like 1(OP9-DL1), which provides a uniform two-dimensional environment to the differentiating thymocytes (9). OP9-DL1 culture system has been reported to support differentiation of progenitors isolated from murine fetal liver (10), adult bone marrow (BM) (11, 12), and human umbilical cord blood and pediatric BM (13, 14).

There has been limited success in generating fully mature T cells from adult human HPC using the OP9-DL1 culture system (13, 15). We have recently shown that CD34 HPC from adult BM display a slower T cell development kinetic than that of fetal and cord blood origins using a lentiviral vector (LV) engineered OP9-DL1 (LmDL1) culture system (16). Proof-of-principle study of retrovirus-mediated transfer of human CD8 T cell receptor (TCR) into human HPC of umbilical cord blood origin or postnatal thymus with the OP9-DL1 culture system has been demonstrated (17, 18). Without an adult T cell development system to produce human leukocyte antigen (HLA)-matched T cells from the patient's own HPC, the latter approach is faced with the challenge of allogeneic transplantation (19).

### SUMMARY

The present addresses at least three limitations of previously utilized in vitro adult human T cell development systems: the limited expansion of preT cells, the inefficient differentiation to double positive (DP) stage and the lack of positive selection and lineage commitment. The inventors have developed an improved system using engineered stromal cells expressing DL1, Flt3-L and/or IL-7, which can enhance preT cell expansion from CD34 HPC. Remarkably, the inventors have discovered that continuous IL-7 signaling impairs further differentiation of immature single positive (ISP) thymocytes into DP thymocytes, thus rendering the developing lymphocytes functionally immature. The process of positive selection is highly regulated by IL-7 receptor (IL-7R) and TCR signals. Interestingly, upon ablation of IL-7R signals and further TCR engagement, positive selection and lineage commitment into CD4 T cells can occur in vitro. Moreover, the inventors demonstrate herein that these CD4 T cells are functionally mature. The advent of a simple in vitro culture system for the generation of functional CD4 T cells from adult human HPC enables a number of translational immunotherapeutic strategies.

### BRIEF DESCRIPTION OF THE DRAWINGS

FIG. 1. Lentiviral vector-modified mouse fetal stromal cell lines. (A) Lentiviral vector constructs. (B) ELISA analysis of IL-7 secretion by LmDL1 and LmDLFL7 cells. (C) Flow cytometry analysis of surface expression of mouse delta like-1 (DL1). (D) Flow cytometry analysis of Flt3L expression of lentiviral vector-modified stromal cell line LmDL1-FL and LmDL1-FL7.

FIG. 2. Lentiviral vector-modified LmDL1-FL7 stromal cells support increased expansion of early T lymphocytes (A) Kinetics of T cell development of adult BM CD34⁺ HPC cultured on LmDL1 supplemented with IL-7 and Flt3L, or on LmDL1-FL7. The developing HPC were sampled from the cocultures on different days as indicated, stained with anti-CD4 and anti-CD8 antibodies, and analyzed with flow cytometry. (B) CD3 and TCRαβ expression kinetics of adult BM CD34⁺ HPC cultured on LmDL1 supplemented with IL-7 and Flt3L, or on LmDL1-FL7. (C) Proliferation curve of differentiating T cells on LmDL1 supplemented with IL-7 and Flt3L, or on LmDL1-FL7. (D) Flow cytometry analysis of T cell maturation markers and nuclear Ki67 after two weeks of anti-CD3/CD28 stimulation from the day 42 coculture. PBMCs (non-stimulated) were used as a control.

FIG. 3. Mature CD4 but not CD8 T cell development from the improved in vitro culture system (A) The experimental design. Growth curve for adult BM CD34⁺ HPC were cultured on LmDL1-FL7 for 24 days and then transferred to LmDL1-FL culture. (B) Flow cytometry analysis of expression kinetics of CD8, CD4, CD3 and TCRαβ. (C) Adult BM CD34⁺ HPC were cultured on LmDL1-FL7 for 24 days and then transferred to LmDL1-FL culture. On day 42, the cells were stimulated and cultured for 14 days before further analysis. Flow cytometry analysis of maturation markers and nuclear Ki67 was performed. PBMCs stimulated under the same condition as above, were used as a control.

FIG. 4. In vitro derived CD4 T cells are functional with a restricted Vβ repertoire (A) T cells stimulated for two weeks were re-stimulated with PMA and ionomycin for 5-6 hours, and stained with antibodies detecting immune effector cytokines and proteins. After removal of IL-7, the T lymphocytes derived from two independent donor BM CD34⁺ HPC in the LmDL1-FL7/L-mDL1-FL cocultures were capable of producing IFN-γ, IL-4, and IL-17, expressed FoxP3 as well as upregulated CD25. Normal PBMC and a primary single cell-derived CD4 T cell clone were included as controls. (B) The Vβ repertoire of in vitro derived T lymphocytes from three different adult bone marrow CD34⁺ HPC donors appeared to be narrow and skewed as compared with a control adult PBMC.

FIG. 5. The improved in vitro T cell development system is capable of generating mature CD4 T cells from adult human HPC. The top diagram illustrated the lack of functional T cell development from the DL1, Flt3L and IL-7 T cell development coculture system. The bottom diagram shows that with lentiviral vector-engineered coexpression of DL1, Flt3L and IL-7, plus the intermittent removal of IL-7, increased amount of mature and functional CD4 T cells are generated.

FIG. S1-3 (S3) Flow cytometric analysis shows that T cell precursors (cultured on OP9FL7 day 42) express high levels of HLA class I and low level of HLA DR DQ DP as compared to stimulated PBMC control. (S1) CD3_{ε} analysis shows that the CD8 cells do express CD3_{ε} chain of the T cell receptor complex similar to the controls, they low level of GATA3 a CD4 lineage marker, and they express PU.1 suggesting arrest in immature stage of differentiation.

### DETAILED DESCRIPTION

Adult bone marrow-derived hematopoietic stem cells (HSCs) are progenitors to all lineages of functional immune cells. However, the molecular signals necessary to direct the full differentiation of HSCs to mature T cells remain obscure. A mouse embryonic stromal cell line engineered to express Delta-like 1 (OP9-DL1), has been reported to support early T cell differentiation but not full maturation of T lymphocytes starting from adult bone marrow derived CD34+ HSCs. There has been limited success in generating mature CD4 T lymphocytes independent of thymus. According to one embodiment, the invention pertains to a viral vector-modified culture system that can support differentiation of adult human CD34+ HSC to fully mature CD4 T lymphocytes in vitro. The engineered stromal cell line expressing DL1, interleukin-7 (IL-7), and FMS-like tyrosine kinase 3 ligand (FL) supports expansion of early differentiated T cells. The continuous IL-7 signaling, however, led to differentiation arrest during immature single positive (ISP) CD8 stage. The inventors solved this problem by a combination approach through temporary termination of IL-7 receptor signaling and activation of CD3/CD28 signaling pathway. This modification resulted in the production of mature CD4 T cells that were able to produce effector cytokines including IFN-γ and TNF-α upon stimulation.

According to one embodiment, the invention pertains to a culture system that can support differentiation of adult human CD34+ hematopoietic stem cells (HSCs) to fully mature CD4 T lymphocytes in vitro.

According to a more specific embodiment, the invention pertains to culturing HSCs in the presence of IL-7 and terminating the subjecting of the cells to IL-7 at a certain window of time over the course of development. In an even more specific embodiment, HSCs are co-cultured with cells, such as OP-9 stromal cells, expressing IL-7, mDL1, and Flt3L (typically by transfection with a viral vector, such as lentivirus) for a period of between 14-24 days. At a time between 14-30 days, the HSCs are no longer subjected to IL-7. The HSCs are later subjected to TCR stimulation. The HSCs develop into fully mature and functional CD4 T cells.

The presently disclosed subject matter also provides methods for inducing an anti-tumor immune response in a subject. In some embodiments, the methods comprise administering to the subject a composition comprising a plurality of T cells and one or more pharmaceutically acceptable carriers or excipients. In some embodiments, the anti-tumor immune response is sufficient to (a) prevent occurrence of a tumor in the subject; (b) delay occurrence of a tumor in the subject; (c) reduce a rate at which a tumor develops in the subject; (d) prevent recurrence of a tumor in the subject; (e) suppress growth of a tumor in a subject; or (f) combinations thereof. In some embodiments, the anti-tumor immune response comprises a cytotoxic T cell response against an antigen present in or on a cell of the tumor. In some embodiments, the cytotoxic T cell response is mediated by CD8+ T cells.

The presently disclosed compositions and methods can also be employed as part of a multi-component anti-tumor and/or anti-cancer treatment modality. In some embodiments, the presently disclosed methods further comprise providing to the subject an additional anti-cancer therapy selected from the group consisting of radiation, chemotherapy, surgical resection, immunotherapy, and combinations thereof. In some embodiments, the additional anti-cancer therapy is provided to the subject at a time prior to, concurrent with, subsequent to, or combinations thereof, the administering step. In some embodiments, the additional anti-cancer therapy is provided prior to the administering step and the composition is administered as an adjuvant therapy.

The presently disclosed compositions and methods can be employed for the prevention and/or treatment of any tumor and/or any cancer. In some embodiments, the cancer is selected from the group consisting of bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments, the cancer comprises a lung carcinoma.

The presently disclosed compositions and methods can be employed for prevention and/or treatment of a tumor and/or a cancer in any subject. In some embodiments, the subject is a mammal. In some embodiments, the mammal is a human.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

Following long-standing patent law tradition, the terms “a”, “an”, and “the” are meant to refer to one or more as used herein, including the claims. For example, the phrase “a cell” can refer to one or more cells. Also as used herein, the term “another” can refer to at least a second or more.

The term “about”, as used herein when referring to a measurable value such as an amount of weight, time, dose (e.g., a number of cells), etc., is meant to encompass variations of in some embodiments .+−.20%, in some embodiments .+−.10%, in some embodiments, .+−.5%, in some embodiments .+−.1%, and in some embodiments .+−.0.1% from the specified amount, as such variations are appropriate to perform the disclosed methods.

As used herein, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used herein, the phrases “treatment effective amount”, “therapeutically effective amount”, “treatment amount”, and “effective amount” are used interchangeably and refer to an amount of a composition (e.g., a plurality of ES cells and/or other pluripotent cells in a pharmaceutically acceptable carrier or excipient) sufficient to produce a measurable response (e.g., a biologically or clinically relevant response in a subject being treated). For example, actual dosage levels of CD4 T cells in the compositions of the presently disclosed subject matter can be varied so as to administer a sufficient number of CD4 T cells to achieve the desired immune response for a particular subject. The selected dosage level will depend upon several factors including, but not limited to the route of administration, combination with other drugs or treatments, the severity of the condition being treated, and the condition and prior medical history of the subject being treated.

As used herein, the term IL-7 means a known IL-7 molecule or a polypeptide having at least 95, 96, 97, or 98 percent identity with IL-7. IL-7 sequences of several different species are well known in the art. Examples of genbank accession nos include AAI10554, BC110553, AAH47698 and BC047698. Percent identity is determined according to conventional techniques and computer programs. For example, percent identity between two sequences, when optimally aligned such as by the programs GAP or BESTFIT (peptides) using default gap weights, or as measured by computer algorithms BLASTX or BLASTP, share the specified identity. Preferably, residue positions which are not identical differ by conservative amino acid substitutions. For example, the substitution of amino acids having similar chemical properties such as charge or polarity are not likely to effect the properties of a protein. Non-limiting examples include glutamine for asparagine or glutamic acid for aspartic acid.

The terms “cancer” and “tumor” are used interchangeably herein and can refer to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder, and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The terms “cancer and “tumor” also encompass solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. As used herein, the terms “cancer and “tumor” are also intended to refer to multicellular tumors as well as individual neoplastic or pre-neoplastic cells. In some embodiments, a tumor is an adenoma and/or an adenocarcinoma, in some embodiments a lung adenoma and/or adenocarcinoma.

The compositions of the presently disclosed subject matter comprise in some embodiments a pharmaceutically acceptable carrier. Any suitable formulation can be used to prepare the disclosed compositions for administration to a subject. In some embodiments, the pharmaceutically acceptable carrier is pharmaceutically acceptable for use in a human.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in some embodiments in the range of 0.1 to 10 mg/ml, in some embodiments about 2.0 mg/ml; and/or mannitol or another sugar, in some embodiments in the range of 10 to 100 mg/ml and in some embodiments about 30 mg/ml; and/or phosphate-buffered saline (PBS).

It should be understood that in addition to the ingredients particularly mentioned above, the formulations of the presently disclosed subject matter can include other agents conventional in the art having regard to the type of formulation in question. Of the possible formulations, sterile pyrogen-free aqueous and non-aqueous solutions can be used.

A composition of the presently disclosed subject matter can be administered to a subject in need thereof in any manner that would be expected to generate and enhance an immune response in the subject. Suitable methods for administration of a composition of the presently disclosed subject matter include, but are not limited to, intravenous (i.v.), intraperitoneal (i.p.), subcutaneous (s.c.), subdermal (s.d.), intramuscular (i.m.), and/or intratumoral injection, and inhalation.

The presently disclosed subject matter methods comprise administering a therapeutically effective dose of a composition of the presently disclosed subject matter to a subject in need thereof. As defined hereinabove, an “effective amount” is an amount of the composition sufficient to produce a measurable response (e.g., enhanced cytolytic and/or cytotoxic response in a subject being treated).

### EXAMPLES

### Example 1

### Increased Expansion of Early T Lymphocytes from Adult Human CD34⁺ Progenitors in a Simplified Lentiviral Vector-Modified Stromal Culture System

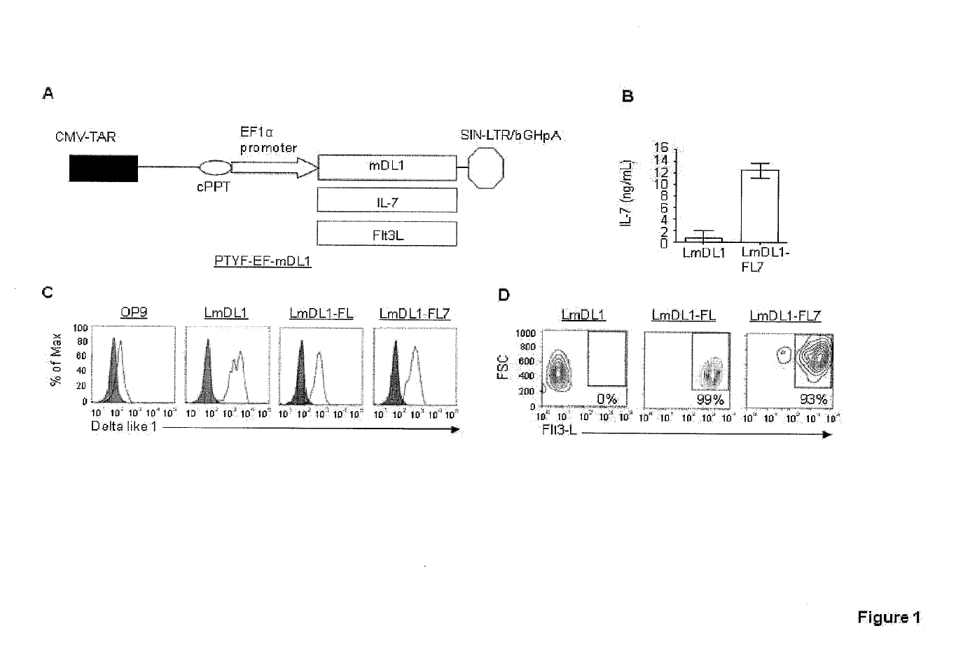

We have previously reported, that a lentiviral vector-modified mouse fetal stromal cell line (LmDL1) expressing mouse delta-like 1 ligand (DL1) can support early T cell differentiation of human CD34⁺ HPC from cord blood, fetal thymus, fetal liver and adult bone marrow (16). To develop a culture system with a stable cytokine environment independent of exogenously added growth factors, we further transduced the LmDL1 cells with lentiviral vectors expressing human Flt3L, or both Flt3L and IL-7, to generate LmDL1-FL and LmDL1-FL7 cell lines, respectively (FIG. 1A). The secretion of IL-7 by LmDL1-FL7 was measured via ELISA to be in the range of 10-14 ng/mL after 48 hours of culture (FIG. 1B). The surface DL1 expression on all three lentiviral vector-transduced cell lines (LmDL1, LmDL1-FL and LmDL1-FL7) was substantially higher than that of the endogenous levels on OP9 as shown by flow cytometry (FIG. 1 C). High surface expression of Flt3L was also illustrated on LmDL1-FL and LmDL1-FL7 cell lines using anti-Flt3-L antibody (FIG. 1 D).

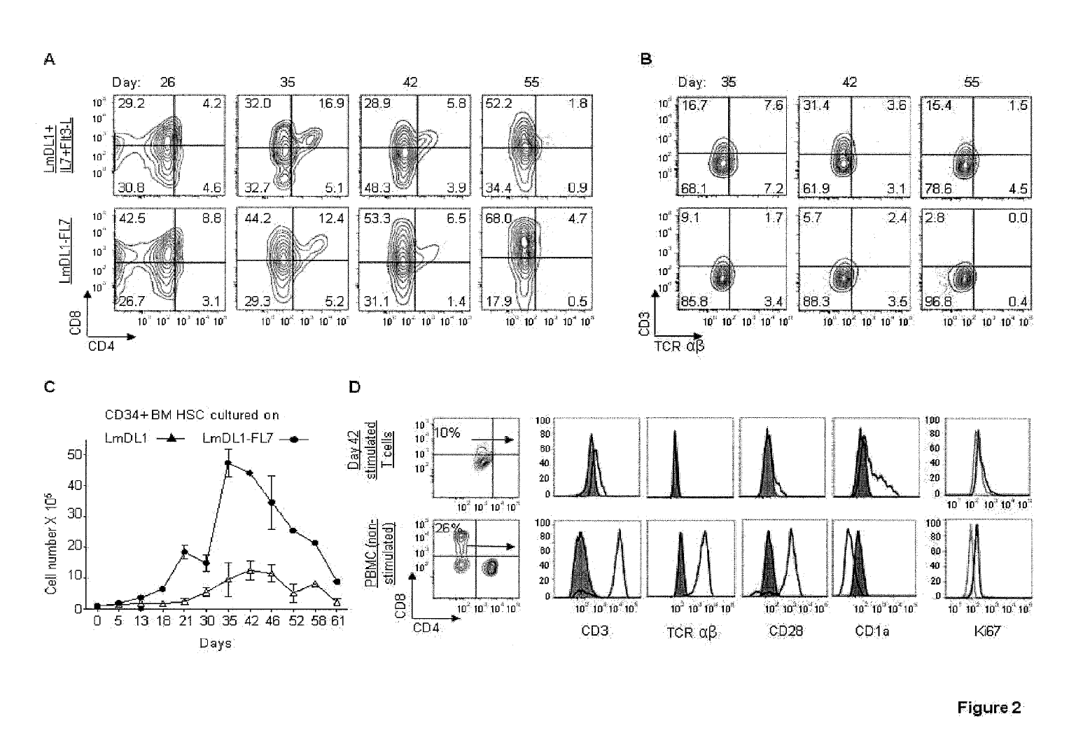

T cell development was demonstrated using highly purified (>97%) adult human CD34 BM cells cultured on LmDL1 cells supplemented with recombinant human IL-7 and Flt3-L, or on LmDL1-FL7 cells without any of the growth factor supplements (FIG. 2). The LmDL1-FL7 culture exhibited a T cell development course similar to that of the LmDL1 culture with slightly higher level of CD8 expression (FIG. 2 A). The CD3 and TCRαβ expression also differed slightly between the two culture systems (FIG. 2 B). Both systems supported development of adult BM CD34⁺ cells into CD3⁻TCRαβ⁻ SP CD8⁺ T cells over the course of 50 to 60 days (FIG. 2). However, we noted a consistent five-fold increase in pre-T cells expansion with the LmDL1-FL7 system as compared with the LmDL1 system (FIG. 2 C). Thus, LmDL1-FL7 cell line supported increased T cell precursor expansion without altering the T cell differentiation potential.

Those skilled in the art will appreciate that other means of transforming cells to express IL-7 can be utilized such as, but not limited to, other viral vectors such as but not limited to Adenoviruses, retroviruses or AAV viruses, or naked DNA. Furthermore, cell types other than fetal stromal cells can be engineered to express IL-7 for co-culturing purposes. Alternatively, IL-7 can be subjected to a target cell type by manually providing to culturing media.

### Example 2

### LmDL1-FL7 Cell Line does not Support Differentiation of BM CD34 HPC into Fully Mature T Cells

The transition of differentiating T cells from double negative (DN) to DP stage and CD4 and CD8 lineages requires Notch signaling as well as pre-TCR signaling (22, 23). The DP T cells depend exclusively on signals downstream of TCR for survival; at this stage they become unresponsive to cytokine induced survival signals (24, 25). We observed that the T cell precursors expressed CD3 but died after about 40 days in the IL-7, Flt3L and Notch signaling coculture (FIG. 2 C). To see if these developing T cells can become mature SP T cells, we provided these T cells with TCR signals by using anti-CD3/anti-CD28 microbeads on day 42 (FIG. 2 D). Following the CD3/CD28 stimulation, the cells expressed low levels of CD8 on the surface. As mature T cells express CD3, TCRαβ and co-stimulatory molecule CD28, and lack CD1a (26), we examined these markers on the developing CD8 SP cells. Antibody staining results illustrated low level of CD3, CD28, undetectable TCRαβ, and marked amount of CD1a (FIG. 2 D), suggesting that these CD8 SP cells were not fully mature. The cultured cells did not show signs of maturation and are non-responsive to TCR signals as demonstrated by nuclear staining for proliferation antigen Ki67 (FIG. 2 D). Similar results were obtained upon stimulating cells obtained from day 50 and day 60 of the coculture (data not shown). Briefly, these results indicate that human BM HPCs cultured with LmDL1-FL7 cells do not develop functional CD8 or CD4 single positive T cells.

### Example 3

### Increased Differentiation from Pre-T to DP T Cells after IL-7 Removal

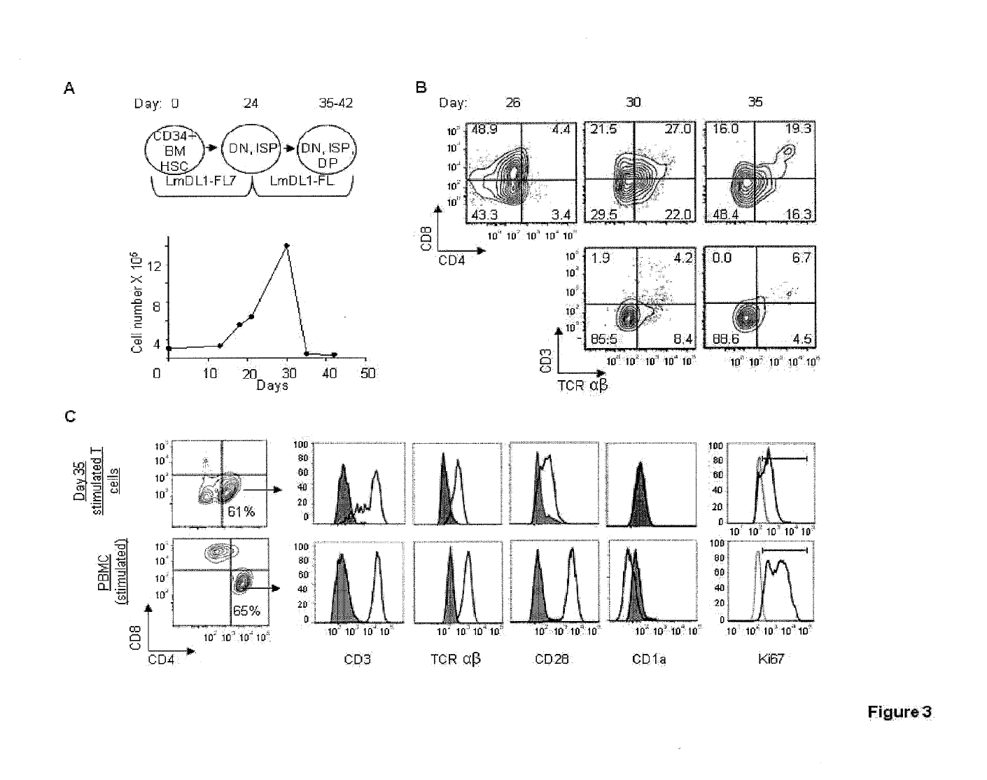

The above results showed that the LmDL1-FL7 culture system does not support differentiation of ISP to DP T cells and full maturation of T cells. In the coculture, only a small percentage of CD3⁺ T cells coexpressed low levels of TCRαβ .suggesting improper TCR rearrangement or processing. FIG. 2 \tilde{B}. Down-regulation of IL-7 receptor signaling is required for further differentiation of pre-T lymphocytes in mice as it interferes with the transcription factors that are required for maturation to CD4CD8 DP stage (27-30). Even though the IL-7 signaling is blocked in DP T cells, these cells reside in a thymic compartment with minimal IL-7 producing cells (31). We hypothesized that efficient T cell differentiation to DP stage in humans might be promoted by removing IL-7 after the appearance of ISP cells. To test this, we cultured adult human BM CD34⁺ cells in LmDL1-FL7 for 24 days and then transferred the cells to LmDL1-FL without IL-7 (FIG. 3 A). After IL-7 removal, we observed a rapid transition into DP stage on day 30 (FIG. 2 A versus 3 B). This transition varied with donors, for some donors the cells became DP on day 35. Along with the appearance of DP cells, co-expression of CD3 and TCRαβ high population was detected, suggesting that these cells underwent positive selection soon after the removal of IL-7. Interestingly, further differentiation along this pathway led to arrested proliferation and cell death (FIG. 3 A).

### Example 4

### Commitment to CD4 T Cell Lineage can be Achieved Upon TCR Stimulation of the IL-7-Deprived Differentiating T Cells

T cell lineage commitment requires cytokine and co-receptor signals (24). We hypothesized that the IL-7-deprived DP T cells will undergo lineage commitment when given a TCR signal. When the CD3 and TCRαβ co-expression was detected between day 30-42 (donor variation), we stimulated the IL-7 deprived T cell precursors with anti-CD3/anti-CD28 microbeads. After TCR signaling, the T cell proliferated as illustrated by Ki67 nuclear staining (FIG. 3 C). In addition, the T cells differentiated beyond ISP stage, as demonstrated by the detection of T cell differentiation and maturation marker including CD3, CD28, and TCRαβ but not CD1a (FIG. 3 C, in comparison with similarly stimulated PBMCs). Thus, continued presence of IL-7 prevents further T cell differentiation beyond ISP stage and impairs functional maturation of developing adult human T cells. Furthermore, these in vitro derived mature T cells were mostly CD4 T cells. The removal of IL-7 may bias cell differentiation toward intermediate CD4⁺ T cells as IL-7 signals are required for the development of CD8⁺ T cells. Subsequent TCR signaling could promote the commitment of intermediate CD4⁺CD8⁻ thymocytes into CD4⁺ T cells, as prolonged TCR signaling (or higher intensity and long duration) can block co-receptor reversal to CD8⁺ SP (20, 32).

### Example 5

### Functional Development of CD4 T Cells in the Improved In Vitro Culture System

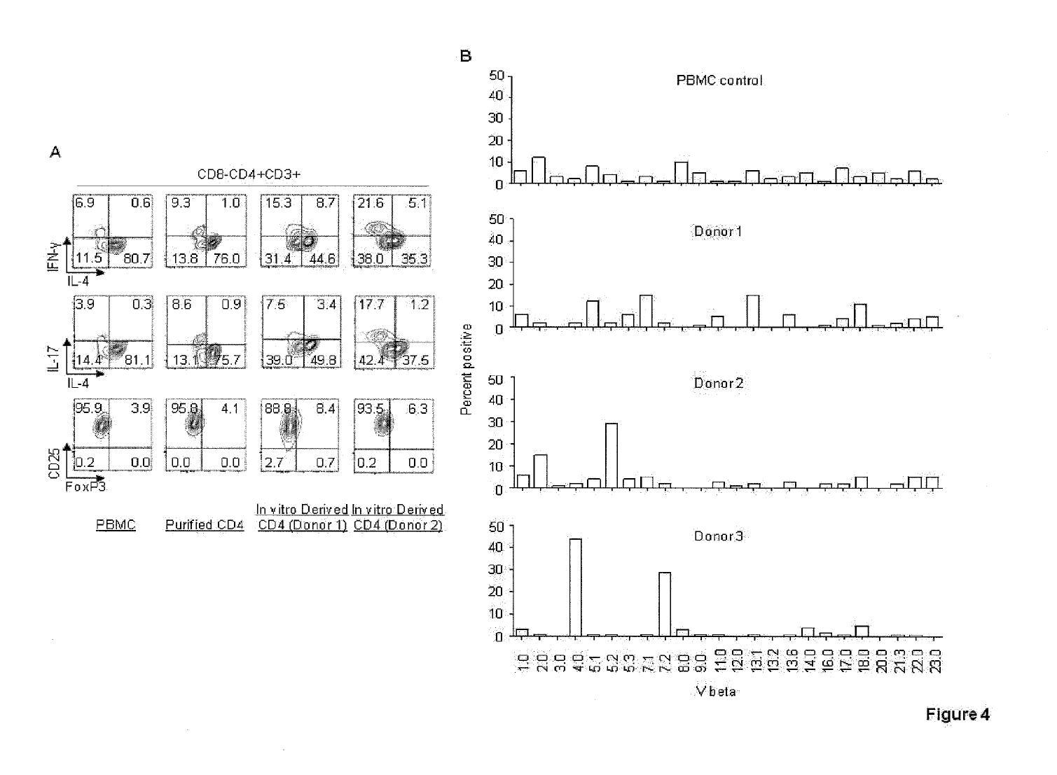

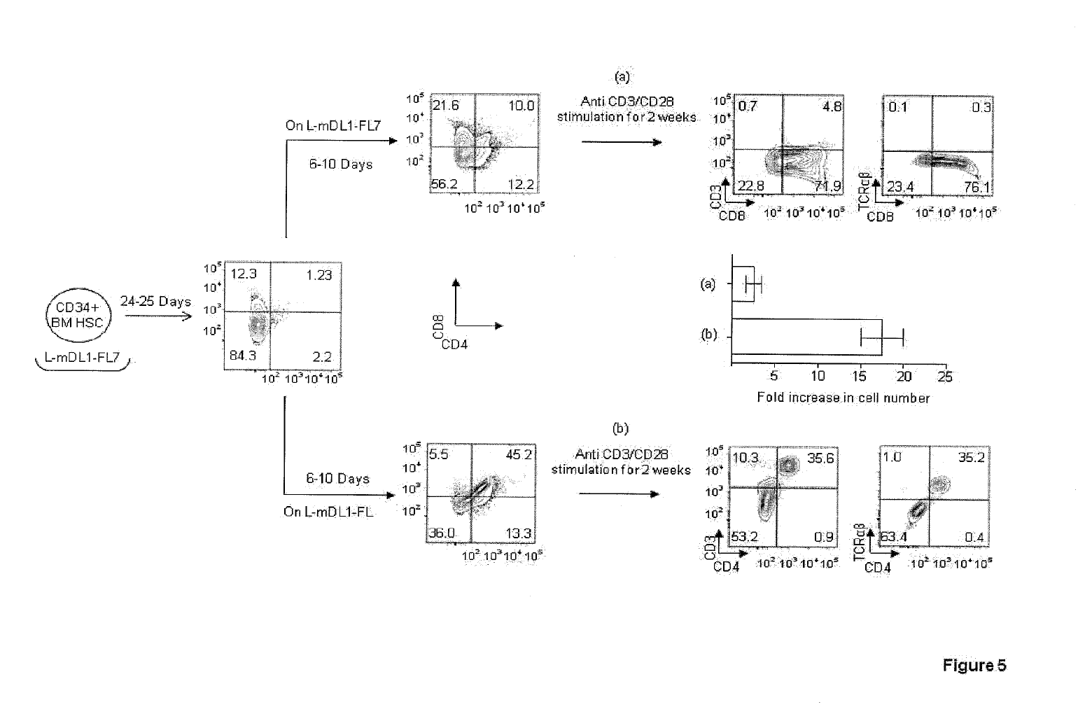

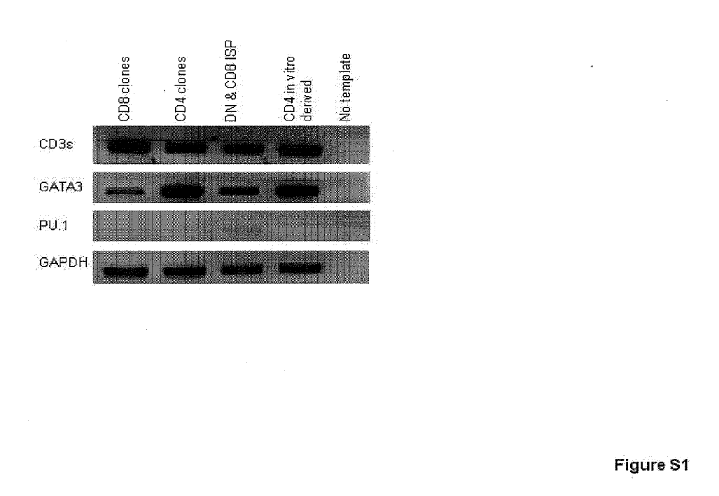

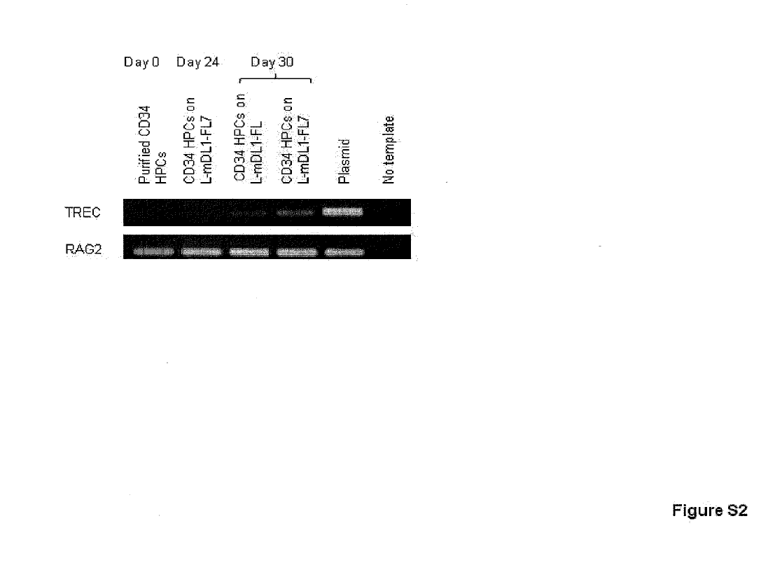

To investigate whether the in vitro derived CD4⁺ T cells could display effector T cell functions, we treated the CD3/CD28 activated, day 42 T cells with PMA and Ionomycin. After 6-8 hr, we analyzed secretion of the effector cytokines IFN-γ, IL-17 and IL-4, by intracellular and surface staining; additionally, we evaluated T regulatory cell related CD25 and FoxP3 expression. The in vitro derived CD4⁺ T lymphocytes, as illustrated from two different donors, were able to secrete IFN-γ, IL-17 and IL-4, and expressed surface CD25 and low levels of intracellular FoxP3 comparable to that of the control PBMC-derived CD4 T cells or a purified primary CD4 T cell clone (FIG. 4 A). The results suggest that these cells are intrinsically programmed to differentiate into various CD4 effector T cell subtypes even in the absence of polarizing culture conditions (33).

### Example 6

### Vβ Repertoire of the In Vitro Generated CD4 SP T Cells is Narrow and Skewed

To evaluate the TCR diversity of the in vitro derived T lymphocytes, Vβ repertoire analysis was performed for 23 Vβ families using IOTest® Beta Mark TCR Vβ Repertoire Kit. The day 42 T cells that expanded into CD4′ SP T cells, were stained with the IOTest® panel of Abs. The in vitro derived CD4⁺ T cells displayed a narrow Vβ usage skewed towards particular Vβ families (FIG. 4 B). For examples, donor 1 displayed a moderately skewed (>10%) usage of Vb5.1, Vb7.1, Vb13.1 and Vb18; donor 2 displayed a skewed usage of Vb2 (15%) and Vb5.2 (29%); donor 3 displayed a highly skewed usage of Vb7.2 (29%) and Vb4 (44%). It appeared that the Vβ repertoires of the in vitro derived T lymphocytes were more restricted than those of normal adult PBMCs.

### Discussion Related to Examples 1-6

Not to be bound by any stated theories, mechanisms or significances, the inventors provide the following discussion related to the results achieved by the Examples 1-6 set forth above:

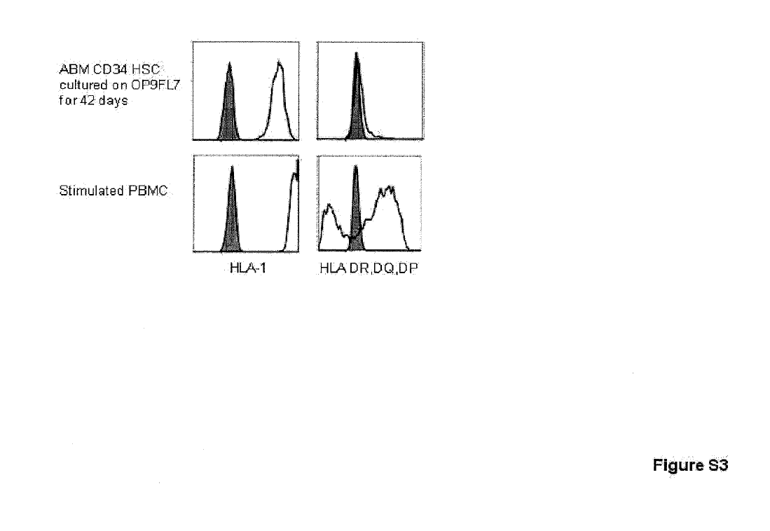

The OP9-DL1 culture system supports development of early T cells from cord blood and fetal liver HPC, yet has not been shown to generate mature T cells from adult human HPC (8-10, 13, 34). Accumulated studies have revealed that the OP9-DL1 system only supports early T cell differentiation to double positive (DP) stage and detailed characterization and functional analysis of these T cells beyond the DP stage have been lacking (10, 13). Although the OP9-DL1 culture system has greatly facilitated human T cell development studies, it remains difficult to produce large number of mature T cells from adult human HPCs in vitro (35). Here the inventors report a modified version of stromal culture system, LmDL1-FL7, which supports increased early T cell expansion from adult CD34⁺ HPC without the needs for exogenous cytokines. The LmDL1-FL7 cell line alone, however, does not support full T cell development from adult human CD34⁺ HPC; rather, the differentiating T cells are arrested at immature single positive (ISP) CD8 T cell stage. This problem is resolved by further modifications of the coculture conditions during DN to DP and SP T cell development stage as summarized in FIG. 5.

None of the published T cell development systems are able to derive fully mature MHC class II-restricted CD4 SP T cells from adult human CD34⁺ HPC (10, 15, 35-38). The culture system described herein is able to support differentiation and maturation of CD4 T cells from adult human CD34⁺ HPC in vitro. The full differentiation of CD34⁺ HPC to CD4 T cells was prompted by CD3/CD28 stimulation of the IL-7-deprived DP T cells. Upon activation, these in vitro developed CD4 T cells secreted IFN-γ, IL-7, IL-4 and expressed CD25 and FoxP3, characteristics of mature and functional T cells. Importantly, the functional response of the in vitro developed T cells is different from those abnormal deregulated CD4 T cells characterized in mice and humans carrying hypomorphic Rag mutations, which are arrested at DN3 stage, abnormally activated and CD3-unresponsive (39-41).

Previous studies in mice suggested that down-regulation of IL-7 receptor signaling in developing T lymphocytes beyond DN3 stage is required to allow efficient differentiation of pro-T into DP T lymphocytes (27, 28, 30, 42, 43). The accumulation of CD8⁺ ISP T lymphocytes from adult HPC in the LmDL1-FL7 coculture most likely reflects a differentiation block before DP stage due to continuous signaling of IL-7, as these cells retain expression of transcription factor PU.1 during early stages of T cell differentiation (FIG. S1 A). Others have shown that IL-7 helps T cell survival and expansion in vitro, but it impedes further progression of ISP to DP T lymphocytes during T cell development in mice (27-29, 42, 44). IL-7R signaling can inhibit expression of transcriptional factors such as transcription factor-1 (TCF-1), lymphoid enhancer-binding factor 1 (LEFT), and the orphan hormone receptor RORγt, critical for ISP to DP transition in mice (28). Our results indicate that the role of IL-7R signaling in T cell development in humans is similar to that in mice as it affects transition from ISP to DP (27-29, 42, 44, 45). It appears that IL-7 does not completely block the transition of developing T cells to the DP stage, rather it renders the ill-differentiated DP T cells unable to respond to TCR stimulation and thus not functional. Further investigation into the role of IL-7 in functional maturation of DP T cells is needed.

In system embodiments described herein, the inventors were able to obtain mature CD4 T cells at the expense of CD8 T cells. The OP9 stromal cells do not express human leukocyte antigen (HLA) class I or class II, it is possible that human thymocytes, however, can provide sufficient class I and class II HLA contacts for maturing DP T cells and induce positive selection (FIG. S1 B) (46, 47). In fact, the expression of MHC class II molecules on human DP T cells is critical for its own positive selection (48). The lineage commitment to CD4 T cells can be explained by the kinetic signaling model, which proposes that DP T cell adopts a CD4 T cell path when receive a positive selecting TCR signal followed by a persistent TCR stimulation; if the TCR signal ceases, the DP cell adopts the CD8 T cell path (20, 24). In certain system embodiments described herein, the inventors provide the IL-7 deprived differentiating T cell precursors with a prolonged TCR signal via anti-CD3/CD28 antibodies, which may account for the CD4 lineage choice.

### Materials and Methods Related to Examples 1-6

Human CD34⁺ cells and cell lines. The adult bone marrow or mobilized peripheral blood CD34⁺ hematopoietic precursor/stem cells (HPC) from normal donors and cord blood CD34⁺ cells were purchased from All Cell Inc. (San Mateo, Calif., USA) or Cambrex (Walkersville, Md.). The mouse fetal stromal cells (OP9) were purchased from the American Type Culture Collection (ATCC, Manassas, Va.). The engineered LmDL1 and LmDL1-FL7 cell lines were generated by transducing cells with lentiviral vectors encoding mouse Delta like 1 (DL1), and DL1, human Flt3L, plus human IL-7, respectively. The stromal cells were maintained in α-MEM (Invitrogen/Gibco BRL, Grand Island, N.Y.) supplemented with 20% fetal bovine serum (FBS, Invitrogen/Gibco BRL) and 1% Penicillin-Streptomycin (Mediatech Inc., Manassas, Va.). IL-7 cytokine secretion was measured by using Human IL-7 ELISA kit. Cell free supernatants were obtained from LmDL1 and LmDLFL7 cells cultured for 48 hrs (80-90% confluent), in a 12 well plate containing 1 ml of media (Ray Biotech, Inc). The samples were read on model 680 microplate reader (Bio-Rad). The surface expression of DL1 and Flt3L was analyzed by flow cytometry with Alexa Fluor 647-conjugated anti-DL1 Ab (Biolegend) and purified anti-Flt3L Ab (Abcam Inc. Cambridge, Mass.) conjugated with zenon-alexa 488 according to manufacturer's instructions (Invitrogen).

LmDL1 stromal cell—CD34⁺ HPC coculture. The CD34⁺ HPC were seeded into 24-well-plate at 1×10⁵ cells/well containing a confluent monolayer of LmDL1 or LmDL1-FL7 cells. The cocultures were maintained in complete medium starting from day 1, consisting of α-MEM with 20% FBS and 1% Penicillin-Streptomycin, supplemented with 5 ng/ml IL-7 (PeproTech, Inc. Rocky Hill, N.J.) and 5 ng/ml Flt3L (PeproTech, Inc.) as indicated. The cocultures were replenished with new media every 2-3 days. The cells in suspension were transferred to a new confluent stromal monolayer once the monolayer began to differentiate or when developing cells reach 80-90% confluent. The cells were transferred by vigorous pipetting, followed by filtering through a 70 μm filter (BD/Falcon, BD Biosciences, Sparks, Md.) and centrifugation at 250 g, at room temperature for 10 min. The cell pellet was transferred to a fresh confluent monolayer. The cells were harvested at the indicated time points during the T cell development for analysis.

Monoclonal antibodies and flow cytometry. The antibodies used for surface staining included CD4 (clone RPA-T4, PE, FITC, PE-Cy7 and Pacific Blue), CD8 (clone RPA-T8 PE, FITC, PE-Cy7 and Pacific Blue), CD3 (clone SK7, PE-Cy7), TCRαβ (clone T10B9.1A-31, FITC) were from BD biosciences, San Jose, Calif. Cells were first washed with PBS plus 2% FBS and blocked with mouse and human serum at 4° C. for 30 min. For each antibody staining, cells were incubated with antibodies per manufacturer's instructions. For each fluorochrome-labeled Ab used, appropriate isotype control was included. After antibody staining, the cells were washed twice and fixed with 2% para-formaldehyde. Data was acquired using BD FACS Diva software (version 5.0.1), on a BD FACSAria and analyzed using the Flowjo software (version 7.1.3.0, Tree Star, Inc. Pasadena, Tex.).

T cell stimulation by anti-CD3/CD28 beads. To stimulate naïve T cells, a protocol for long term stimulation was followed using anti-CD3/CD28 beads (Dynal/Invitrogen, San Diego, Calif.) per manufacturer's instructions. The cells and the beads were mixed and plated into a 96 well plate at 37° C. for 2-3 days in X-vivo 20 (BioWhittaker, Cambrex, Walkersville, Md.) media, on day 3 12.5U of IL-2, 5 ng/ml of IL-7 and 20 ng/ml of IL-15 were added and the cells were cultured for additional 11-12 days. Surface staining was done as described above using the following antibodies CD4 (clone RPA-T4, PE, FITC, PE-Cy7 and Pacific Blue), CD8 (clone RPA-T8 PE, FITC, PE-Cy7 and Pacific Blue), CD3 (clone SK7, PE-Cy7), TCRαβ (clone T10B9.1A-31, FITC), CD1a (clone HI149, APC) were from BD biosciences. CD28 (clone CD28.2, APC) was from eBioscience Inc. (San Diego, Calif.). Intracellular staining was done using anti-Ki67 (clone B56, FITC), and isotype IgG₁κ from BD biosciences. Intracellular staining was done using anti-Ki67 FITC, and isotype IgG₁κ (BD Biosciences). Intracellular staining was performed using BD cytofix/cytoperm kit, according to the manufacturer's protocol.

Effector function analysis of in vitro generated CD4⁺ T cells. The CD3/CD28 expanded CD4 T cells were stimulated with PMA and Ionomycin (Sigma-Aldrich, St. Louis, Mo.), and analyzed for the release of IFN-γ, IL-4 and IL-17. Briefly the cells were incubated with 25 ng/ml PMA and 1 μg/ml Ionomycin for one hour followed by addition of 6 μg/ml monensin (Sigma-Aldrich) to inhibit Golgi-mediated cytokine secretion. After 4-5 hours of incubation the cells were harvested and surface stained for CD4 (clone RPA-T4, Pacific blue, CD8 (clone SK1, APC-Cy7), CD3 (clone SK7 PE-Cy7), CD25 (clone M-A251, PE) and intracellular stained for IFN-γ-(clone 25723.11, FITC), IL-4-(clone MP425D2, APC, FOXP3 (clone PCH101, Alexa 647) were from BD Biosciences, IL-17 (clone 64CAP17, PE) was from e-Biosciences. The data were collected by flow cytometry using BD FACSAria and analyzed using Flowjo.

The Vβ repertoire analysis of in vitro derived CD4⁺ T cells. The Vβ repertoire of in vitro developed T lymphocytes was analyzed by using IOTest® Beta Mark TCR Vβ Repertoire Kit (Beckman Coulter, Fullerton, Calif.). Staining for 24 Vβ families was performed according to manufacturer's protocol.

Materials and Method related to Supplemental Figures.

Antibodies

Antibodies used were, HLA Class I (clone TU149, PE) from Clatag, HLA DR DQ DP (clone TU39, FITC) from BD biosciences.

RT-PCR

RNA was harvested from CD8, CD4 single cell clones, in vitro developed DN +CD8, in vitro developed CD4 T cells using TRI Reagent (Sigma-Aldrich). 1 ug RNA was reverse transcribed into cDNA by using Two-step AMV RT-PCR kit (Gene choice, MD). The following primers were used for the PCR reaction GAPDH-F-5′CCG ATG GCA AAT TCG ATG GC 3′ and R-5′ GAT GAC CCT TTT GGC TCC CC 3′, PU.1F-5′ TGG AAG GGT TTC CCC TCG TC 3′ and R-5′ TGC TGT CCT TCA TGT CGC CG 3′, CD3e F-5′ TGA AGC ATC ATC AGT AGT CAC AC 3′ and R-5′ GGC CTC TGT CAA CAT TTA CC 3′, GATA-3 F-5′ GAC GAG AAA GAG TGC CTC AAG 3′ and R-5′ TCC AGA GTG TGG TTG TGG TG 3′. After 30 cycles of amplification (95° C. for 30 seconds, 55° C. for 30 seconds, and 72° C. for 60 seconds), PCR products were separated on a 2% agarose gel.

### REFERENCES

The disclosures of all references cited herein, including related applications, are incorporated in their entirety to the extent non inconsistent with the teachings herein.

The following list includes the full cites of references described above and additional related references.
1. Dudley, M. E., and S. A. Rosenberg. 2003. Adoptive-cell-transfer therapy for the treatment of patients with cancer. Nat Rev Cancer 3:666-675.
2. Gitelson, E., C. Hammond, J. Mena, M. Lorenzo, R. Buckstein, N. L. Berinstein, K. Imrie, and D. E. Spaner. 2003. Chronic lymphocytic leukemia-reactive T cells during disease progression and after autologous tumor cell vaccines. Clin Cancer Res 9:1656-1665.
3. Gattinoni, L., D. J. Powell, Jr., S. A. Rosenberg, and N. P. Restifo. 2006. Adoptive immunotherapy for cancer: building on success. Nat Rev Immunol 6:383-393.
4. Plum, J., M. De Smedt, M. P. Defresne, G. Leclercq, and B. Vandekerckhove. 1994. Human CD34+ fetal liver stem cells differentiate to T cells in a mouse thymic microenvironment. Blood 84:1587-1593.
5. Yeoman, H., R. E. Gress, C. V. Bare, A. G. Leary, E. A. Boyse, J. Bard, L. D. Shultz, D. T. Harris, and D. DeLuca. 1993. Human bone marrow and umbilical cord blood cells generate CD4+ and CD8+ single-positive T cells in murine fetal thymus organ culture. Proceedings of the National Academy of Sciences of the United States of America 90:10778-10782.
6. Poznansky, M. C., R. H. Evans, R. B. Foxall, I. T. Olszak, A. H. Piascik, K. E. Hartman, C. Brander, T. H. Meyer, M. J. Pykett, K. T. Chabner, S. A. Kalams, M. Rosenzweig, and D. T. Scadden. 2000. Efficient generation of human T cells from a tissue-engineered thymic organoid. Nature biotechnology 18:729-734.
7. Yeoman, H., D. R. Clark, and D. DeLuca. 1996. Development of CD4 and CD8 single positive T cells in human thymus organ culture: IL-7 promotes human T cell production by supporting immature T cells. Developmental and comparative immunology 20:241-263.
8. Robinson, K. L., J. Ayello, R. Hughes, C. van de Ven, L. Issitt, J. Kurtzberg, and M. S. Cairo. 2002. Ex vivo expansion, maturation, and activation of umbilical cord blood-derived T lymphocytes with IL-2, IL-12, anti-CD3, and IL-7. Potential for adoptive cellular immunotherapy post-umbilical cord blood transplantation. Experimental hematology 30:245-251.
9. Zuniga-Pflucker, J. C. 2004. T-cell development made simple. Nat Rev Immunol 4:67-72.
10. Schmitt, T. M., and J. C. Zuniga-Pflucker. 2002. Induction of T cell development from hematopoietic progenitor cells by delta-like-1 in vitro. Immunity 17:749-756.
11. Schmitt, T. M., R. F. de Pooter, M. A. Gronski, S. K. Cho, P. S. Ohashi, and J. C. Zuniga-Pflucker. 2004. Induction of T cell development and establishment of T cell competence from embryonic stem cells differentiated in vitro. Nature immunology 5:410-417.
12. Huang, J., K. P. Garrett, R. Pelayo, J. C. Zuniga-Pflucker, H. T. Petrie, and P. W. Kincade. 2005. Propensity of adult lymphoid progenitors to progress to DN2/3 stage thymocytes with Notch receptor ligation. J Immunol 175:4858-4865.
13. De Smedt, M., I. Hoebeke, and J. Plum. 2004. Human bone marrow CD34+ progenitor cells mature to T cells on OP9-DL1 stromal cell line without thymus microenvironment. Blood cells, molecules & diseases 33:227-232.
14. La Motte-Mohs, R. N., E. Herer, and J. C. Zuniga-Pflucker. 2005. Induction of T-cell development from human cord blood hematopoietic stem cells by Delta-like 1 in vitro. Blood 105:1431-1439.
15. Martinic, M. M., M. F. van den Broek, T. Rulicke, C. Huber, B. Odermatt, W. Reith, E. Horvath, R. Zellweger, K. Fink, M. Recher, B. Eschli, H. Hengartner, and R. M. Zinkernagel. 2006. Functional CD8+ but not CD4+ T cell responses develop independent of thymic epithelial MHC. Proceedings of the National Academy of Sciences of the United States of America 103:14435-14440.
16. Patel, E., B. Wang, L. Lien, L.-J. Yang, J. S. Moreb, and L.-J. Chang. 2008. Diverse T cell differentiation potentials of human fetal thymus, fetal liver, cord blood and adult bone marrow CD34 cells on lentiviral Delta-like 1-modified mouse stromal cells. Immunology (in press)
17. Zhao, Y., A. D. Bennett, Z. Zheng, Q. J. Wang, P. F. Robbins, L. Y. Yu, Y. Li, P. E. Molloy, S. M. Dunn, B. K. Jakobsen, S. A. Rosenberg, and R. A. Morgan. 2007. High-affinity TCRs generated by phage display provide CD4+ T cells with the ability to recognize and kill tumor cell lines. J Immunol 179:5845-5854.
18. van Lent, A. U., M. Nagasawa, M. M. van Loenen, R. Schotte, T. N. M. Schumacher, M. H. M. Heemskerk, H. Spits, and N. Legrand. 2007. Functional Human Antigen-Specific T Cells Produced In Vitro Using Retroviral T Cell Receptor Transfer into Hematopoietic Progenitors. J Immunol 179:4959-4968.
19. Heemskerk, M. H., R. S. Hagedoorn, M. A. van der Hoorn, L. T. van der Veken, M. Hoogeboom, M. G. Kester, R. Willemze, and J. H. Falkenburg. 2007. Efficiency of T-cell receptor expression in dual-specific T cells is controlled by the intrinsic qualities of the TCR chains within the TCR-CD3 complex. Blood 109:235-243.
20. Brugnera, E., A. Bhandoola, R. Cibotti, Q. Yu, T. I. Guinter, Y. Yamashita, S. O. Sharrow, and A. Singer. 2000. Coreceptor reversal in the thymus: signaled CD4+8+ thymocytes initially terminate CD8 transcription even when differentiating into CD8+ T cells. Immunity 13:59-71.
21. Yu, Q., B. Erman, A. Bhandoola, S. O. Sharrow, and A. Singer. 2003. In vitro evidence that cytokine receptor signals are required for differentiation of double positive thymocytes into functionally mature CD8+ T cells. The Journal of experimental medicine 197:475-487.
22. Garbe, A. I., A. Krueger, F. Gounari, J. C. Zuniga-Pflucker, and H. von Boehmer. 2006. Differential synergy of Notch and T cell receptor signaling determines alphabeta versus gammadelta lineage fate. The Journal of experimental medicine 203:1579-1590.
23. Laky, K., and B. J. Fowlkes. 2008. Notch signaling in CD4 and CD8 T cell development. Curr Opin Immunol
24. Singer, A., S. Adoro, and J. H. Park. 2008. Lineage fate and intense debate: myths, models and mechanisms of CD4- versus CD8-lineage choice. Nat Rev Immunol 8:788-801.
25. DeJarnette, J. B., C. L. Sommers, K. Huang, K. J. Woodside, R. Emmons, K. Katz, E. W. Shores, and P. E. Love. 1998. Specific requirement for CD3epsilon in T cell development. Proceedings of the National Academy of Sciences of the United States of America 95:14909-14914.
26. Res, P., B. Blom, T. Hori, K. Weijer, and H. Spits. 1997. Downregulation of CD1 marks acquisition of functional maturation of human thymocytes and defines a control point in late stages of human T cell development. The Journal of experimental medicine 185:141-151.
27. Yasuda, Y., A. Kaneko, I. Nishijima, S. Miyatake, and K. Arai. 2002. Interleukin-7 inhibits pre-T-cell differentiation induced by the pre-T-cell receptor signal and the effect is mimicked by hGM-CSF in hGM-CSF receptor transgenic mice. Immunology 106:212-221.
28. Yu, Q., B. Erman, J. H. Park, L. Feigenbaum, and A. Singer. 2004. IL-7 receptor signals inhibit expression of transcription factors TCF-1, LEF-1, and RORgammat: impact on thymocyte development. The Journal of experimental medicine 200:797-803.
29. Hassan, J., and D. J. Reen. 1998. IL-7 promotes the survival and maturation but not differentiation of human post-thymic CD4+ T cells. Eur J Immunol 28:3057-3065.
30. David-Fung, E. S., M. A. Yui, M. Morales, H. Wang, T. Taghon, R. A. Diamond, and E. V. Rothenberg. 2006. Progression of regulatory gene expression states in fetal and adult pro-T-cell development. Immunological reviews 209:212-236.
31. Zamisch, M., B. Moore-Scott, D. M. Su, P. J. Lucas, N. Manley, and E. R. Richie. 2005. Ontogeny and regulation of IL-7-expressing thymic epithelial cells. J Immunol 174:60-67.
32. Suzuki, H., Y. Shinkai, L. G. Granger, F. W. Alt, P. E. Love, and A. Singer. 1997. Commitment of immature CD4+8+ thymocytes to the CD4 lineage requires CD3 signaling but does not require expression of clonotypic T cell receptor (TCR) chains. The Journal of experimental medicine 186:17-23.
33. O'Garra, A. 1998. Cytokines induce the development of functionally heterogeneous T helper cell subsets. Immunity 8:275-283.
34. Schmitt, T. M., and J. C. Zuniga-Pflucker. 2006. T-cell development, doing it in a dish. Immunological reviews 209:95-102.
35. Offner, F., T. Kerre, M. De Smedt, and J. Plum. 1999. Bone marrow CD34 cells generate fewer T cells in vitro with increasing age and following chemotherapy. British journal of haematology 104:801-808.
36. Freedman, A. R., H. Zhu, J. D. Levine, S. Kalams, and D. T. Scadden. 1996. Generation of human T lymphocytes from bone marrow CD34+ cells in vitro. Nature medicine 2:46-51.
37. McKean, D. J., C. J. Huntoon, M. P. Bell, X. Tai, S. Sharrow, K. E. Hedin, A. Conley, and A. Singer. 2001. Maturation versus death of developing double-positive thymocytes reflects competing effects on Bcl-2 expression and can be regulated by the intensity of CD28 costimulation. J Immunol 166:3468-3475.
38. Galic, Z., S. G. Kitchen, A. Kacena, A. Subramanian, B. Burke, R. Cortado, and J. A. Zack. 2006. T lineage differentiation from human embryonic stem cells. Proceedings of the National Academy of Sciences of the United States of America 103:11742-11747.
39. Sobacchi, C., V. Marrella, F. Rucci, P. Vezzoni, and A. Villa. 2006. RAG-dependent primary immunodeficiencies. Human mutation 27:1174-1184.
40. Khiong, K., M. Murakami, C. Kitabayashi, N. Ueda, S. Sawa, A. Sakamoto, B. L. Kotzin, S. J. Rozzo, K. Ishihara, M. Verella-Garcia, J. Kappler, P. Marrack, and T. Hirano. 2007. Homeostatically proliferating CD4 T cells are involved in the pathogenesis of an Omenn syndrome murine model. The Journal of clinical investigation 117:1270-1281.
41. Marrella, V., P. L. Poliani, C. Sobacchi, F. Grassi, and A. Villa. 2008. Of Omenn and mice. Trends in immunology 29:133-140.
42. Wang, H., L. J. Pierce, and G. J. Spangrude. 2006. Distinct roles of IL-7 and stem cell factor in the OP9-DL1 T-cell differentiation culture system. Experimental hematology 34:1730-1740.
43. Swainson, L., E. Verhoeyen, F. L. Cosset, and N. Taylor. 2006. IL-7R alpha gene expression is inversely correlated with cell cycle progression in IL-7-stimulated T lymphocytes. J Immunol 176:6702-6708.
44. El Kassar, N., P. J. Lucas, D. B. Klug, M. Zamisch, M. Merchant, C. V. Bare, B. Choudhury, S. O. Sharrow, E. Richie, C. L. Mackall, and R. E. Gress. 2004. A dose effect of IL-7 on thymocyte development. Blood 104:1419-1427.
45. Mazzucchelli, R., and S. K. Durum. 2007. Interleukin-7 receptor expression: intelligent design. Nat Rev Immunol 7:144-154.
46. Traggiai, E., L. Chicha, L. Mazzucchelli, L. Bronz, J. C. Piffaretti, A. Lanzavecchia, and M. G. Manz. 2004. Development of a human adaptive immune system in cord blood cell-transplanted mice. Science (New York, N.Y. 304:104-107.
47. Li, W., M. G. Kim, T. S. Gourley, B. P. McCarthy, D. B. Sant'Angelo, and C. H. Chang. 2005. An alternate pathway for CD4 T cell development: thymocyte-expressed MHC class II selects a distinct T cell population. Immunity 23:375-386.
48. Choi, E. Y., W. S. Park, K. C. Jung, D. H. Chung, Y. M. Bae, T. J. Kim, H. G. Song, S. H. Kim, D. I. Ham, J. H. Hahn, J. Kim, K. Kim, T. S. Hwang, and S. H. Park. 1997. Thymocytes positively select thymocytes in human system. Human immunology 54:15-20

The above-described embodiments and configurations are neither complete nor exhaustive. As will be appreciated, other embodiments of the invention are possible utilizing, alone or in combination, one or more of the features set forth above or described in detail below. In addition, the present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of producing fully mature and functional CD4 T cells from hematopoietic stem cells (HSCs) wherein the method comprises culturing the HSCs under culture conditions to direct development of said HSCs to the functional CD4 T cells, the culture conditions comprising:
culturing the HSCs in the presence of IL-7 for at least 2 weeks, and
terminating subjection of said stem cells to IL-7 at a time somewhere between about 2 weeks to about 4 weeks

2. The method of claim 1, wherein said method comprises terminating subjection of said stem cells to IL-7 at a time somewhere between about 3 weeks to about 4 weeks.

3. The method of claim 1, wherein said method comprises terminating subjection of said stem cells to IL-7 at a time somewhere 20 to 28 days.

4. The method of claim 1, wherein said culturing comprises co-culturing said HSCs with modified fetal stromal cells engineered to express delta-like 1 ligand and IL-7 and/or Flt31.

5. The method of claim 4, wherein said modified fetal stromal cells are mammalian cells.

6. The method of claim 5, wherein said modified fetal stromal cells are of mouse, rat, rabbit, or guinea pig origin.

7. The method of claim 4, wherein said modified fetal stromal cells have been transfected with a vector comprising a polynucleotide that encodes IL-7, or a polypeptide molecule having at least 95 percent identity with said IL-7.

8. The method of claim 7, wherein said vector is a viral vector.

9. The method of claim 8, wherein said vector is a lentiviral vector.

10. A pharmaceutical composition comprising functional CD4 T cells cultured and produced from adult human bone marrow and a pharmaceutically acceptable carrier, excipient, or diluent.

11. A method of treating cancer by administering a therapeutically effective amount of the composition of claim 10 in a patient in need thereof.

12. The method of claim 10, wherein said cancer is melanoma or leukemia.

13. An isolated cell sample of modified fetal stromal cells engineered to express delta-like 1 ligand and IL-7 and/or Flt31.

14. The isolated cell sample of claim 13, wherein said cells are murine, rat, rabbit, or guinea pig cells.


SEQUENCE LISTING (8 sequences)

SEQ ID NO: 1 - DNA, 20 bp, Artificial Sequence
  Description of Artificial Sequence Synthetic primer
ccgatggcaaattcgatggc

SEQ ID NO: 2 - DNA, 20 bp, Artificial Sequence
  Description of Artificial Sequence Synthetic primer
gatgacccttttggctcccc

SEQ ID NO: 3 - DNA, 20 bp, Artificial Sequence
  Description of Artificial Sequence Synthetic primer
tggaagggtttcccctcgtc

SEQ ID NO: 4 - DNA, 20 bp, Artificial Sequence
  Description of Artificial Sequence Synthetic primer
tgctgtccttcatgtcgccg

SEQ ID NO: 5 - DNA, 23 bp, Artificial Sequence
  Description of Artificial Sequence Synthetic primer
tgaagcatcatcagtagtcacac

SEQ ID NO: 6 - DNA, 20 bp, Artificial Sequence
  Description of Artificial Sequence Synthetic primer
ggcctctgtcaacatttacc

SEQ ID NO: 7 - DNA, 21 bp, Artificial Sequence
  Description of Artificial Sequence Synthetic primer
gacgagaaagagtgcctcaag

SEQ ID NO: 8 - DNA, 20 bp, Artificial Sequence
  Description of Artificial Sequence Synthetic primer
tccagagtgtggttgtggtg